(19) United States
(12) Patent Application Publication
(10) Pub. No.: US 2006/0171844 A1
(43) Pub. Date: Aug. 3, 2006

(54) Compact aerosol concentrator for continuous use

(76) Inventors: Constantinos Sioutas, Los Angeles, CA (US); Philip M. Fine, Topanga, CA (US)

Correspondence Address: FISH & RICHARDSON, PC, P.O. BOX 1022, MINNEAPOLIS, MN, 55440-1022, US

(21) Appl. No.: 11/343,787

(22) Filed: Jan. 30, 2006

Related U.S. Application Data
(60) Provisional application No. 60/648,879, filed on Jan. 31, 2005.

(51) Int. Cl. G01N 33/00

(52) U.S. Cl. 422/73

(57) ABSTRACT

Particle detection using a system that enlarges particles, concentrates them, and then dries them to return them to their original sizes. Semiconductor components may be used to maintain better control over the process.

### CROSS REFERENCE TO RELATED APPLICATIONS

This application claims priority to U.S. Provisional Application Serial No. 60/648,879, filed on Jan. 31, 2005. The disclosure of the prior application is considered part of (and is incorporated by reference in) the disclosure of this application.

### FEDERALLY SPONSORED RESEARCH OR DEVELOPMENT

The U.S. Government may have certain rights in this invention pursuant to South Coast AQMD Grant No. 04062.

### BACKGROUND

Detection of aerosol concentration may be used for various purposes.

At least one study has linked the level of ambient particulate matter or PM with adverse health effects. Studies are ongoing to detect the specific physical and/or chemical properties of the particulate matter that are responsible for the adverse health effects. Fast and accurate measures of particulate matter characteristics may be helpful for these features.

Different kinds of particle concentrators have been used to study the characteristics of particulate matter. Previous systems have used virtual impaction, slit virtual impactors, as well as other techniques.

A versatile aerosol concentration enrichment system, or VACES, may be used to detect particles. The VACES system first grows the particles to larger sizes, via supersaturation in water vapor, and then concentrates them by inertial virtual impaction, and returns the particles to their original size by diffusion and drying.

The current VACES configuration consumes, however, significant electrical power, and also has typically required attended operation by experts.

### SUMMARY

The present application describes a special, miniaturized VACES system, with certain structural differences from the prior art. These differences may enable miniaturization and unattended operation.

### BRIEF DESCRIPTION OF THE DRAWINGS

These and other aspects will now be described in detail with reference to the accompanying drawings, wherein:

FIG. 1 shows a block diagram of the overall system.

### DETAILED DESCRIPTION

The device described herein may form particle enriched air that can be used as an elevated exposure atmosphere for exposure studies, or can be used to collect the material. The particle enriched material can also be used in sampling instrumentation, to provide elevated levels of ambient particulate matter. The particle enriched air may also be used for collection of particles in aqueous solutions. The These systems do not significantly alter the physical or chemical properties of the particles.

An embodiment is described of a miniaturized particle enricher. This device can be used for any of the purposes described above. Another embodiment may use the concentrated aerosol stream to form continuous particle stream for a mass spectrometer. The continuous aerosol stream may increase the spectrometer's hit rate or sensitivity.

In this application, a low intake flow rate, for example, less than 1 L per minute, may be used and an unattended 24-hour a day sampling technique may also be used.

The system described herein has a nominal intake flow rate of 30 L per minute, a nominal minor flow rate of 1-1.5 L per minute, and more automated operation.

The system uses humidification of the air stream using a special saturator. The saturator uses a heated moist absorbent material surrounding the intake flow. Cooling is carried out to achieve supersaturation and particle growth. Improved control of temperature, and miniaturization is obtained by using a solid-state thermoelectric chiller. A draining system to a closed vessel removes extra water vapor. In addition, by maintaining better control of the temperature, freezing and ice are reduced.

The particles in the aerosol are caused to increase in size by the supersaturation and freezing. The grown particles are then concentrated using a virtual impactor.

The concentrated particles are then returned to their original size using a diffusion dryer that is filled with silica gel.

As a result of laboratory evaluation, this new system has been found to include near ideal enrichment factors for particles of different compositions, and has also been found to not materially change the particle size distributions.

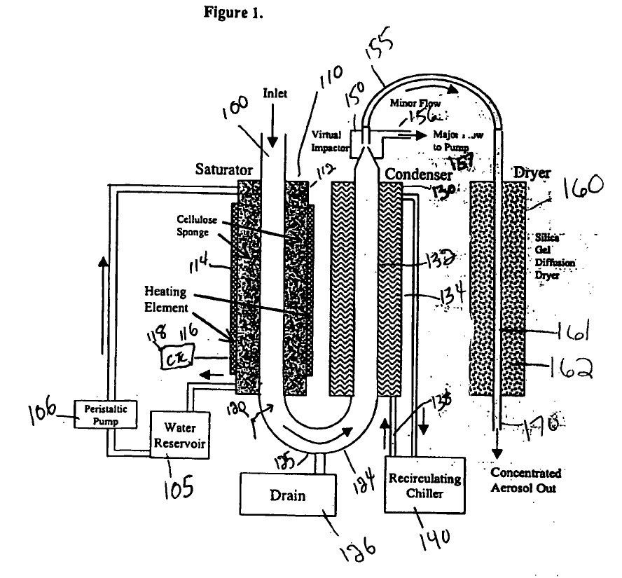

Details of the embodiment are described herein with respect to FIG. 1. The air inlet 100 may be a 2.54 cm inner diameter inlet, and air may travel at 30 L per minute. The system may operate with a small pressure drop, e.g. that of the type associated with standard PM_{2.5}, inlet impactor or cyclone.

The following describes the specific sizes and dimensions of the structure used in this system. However, it should be understood that any of these dimensions may be varied while still be maintained within the teachings herein. In particular, any of these dimensions may be varied by 50%, or more than 50%.

A saturator 110 is located in the path of the inlet air. The saturator is formed of a 2.54 cm inner diameter and 45 cm long circular channel, surrounded by a cellulose sponge 112 contained within an aluminum cylinder 114. A heating tape 116 may be wrapped around the exterior of the saturator, and controlled by a heat controller 118. For example, the heat controller may be a variable transform. A voltage is maintained, such as to heat the air, so that the air leaving the saturator is maintained between 28 and 29° C., and having a relative humidity greater than 90%.

A temperature/humidity probe 120 may be used immediately downstream of the saturator 110 in order to measure the temperature and humidity. In an embodiment, the temperature and humidity probe may be a model 37960,available from Cole-Parmer Instruments of Vernon Hills, Ill..

The saturator is maintained wet using water from water reservoir 105 which is pumped by peristaltic pump 106. A closed system is used, to maintain the pressure differential.

The saturated material flow passes through a section 124 of tubing, which is substantially in a U-shape. The U-shape is formed with a drain 126 at a bottommost portion thereof. The drain forms a gravity fed drain that removes excess condensed water from the saturator 110, (and also from the condenser, which is to be described herein). The drain 126 forms a basin as a closed system to compensate for certain pressure drops. The bottom portion 125 of the U tube 124, where the drain is located, is physically lower than other portions.

After the U section 124, flow passes through the condenser 130. The condenser 130 is formed of a 2.54 cm inner tube 132 surrounded by a 7.62 cm outer tube 134. Both tubes of the condenser are 27 cm long. The air flow passes through the inner tube 132, while the space between the outer tube 134 and inner tube 132 is cooled. In the embodiment, a continuous flow of chilled 1:1 mixture of ethylene glycol to water forms a coolant which fills the space between the inner and outer tubes.

A recirculating chiller 140, circulates the coolant 138 through the space. The chiller 140 is formed of a thermoelectric cooler is run to maintain the temperature of the outer wall of the condenser at −1° C. The thermoelectric chiller may be duty cycle modulated to maintain the desired temperature.

In the embodiment, the chiller may be a Thermocube 300-1D-1-LT, available from solid State cooling systems Pleasant Valley N.Y. Since the thermoelectric cooler may be more easily controlled than other cooling devices, the temperature may be kept at −1° C.: substantially higher than the temperatures used in the larger devices. This may thus may eliminate or minimize any problem of ice buildup on the inner walls.

In operation, the condenser 130 supersaturates the air stream, and particles grow by condensation to a diameter that is above the cut point of the virtual impactor 150. The virtual impactor is a minimized size virtual impactor with a 50% cut point of about 1.5 μ in aerodynamic diameter. Inertial forces are used to concentrate the particle containing droplets in the minor flow 155 of the impactor. The minor flow continues through the conduit 155. The major flow 156 is substantially particle free, and is drawn away by vacuum pump 157. In the embodiment, the vacuum pump may be a model 0523-101Q-G582 DX available from Gast of Benton Harbor Mich..

In the embodiment, the minor flow can range between 0.6 L per minute and 2 L per minute depending on the application of the desired amount of enrichment

The minor flow in conduit 155 is then sent to a dryer 160. The dryer includes an inner tube 161 which is 1.1 cm in diameter and 15 cm in length. The tube is formed of a metal screen surrounded by baked silica gel 162. The gel may be re-baked or changed periodically. The dryer removes the water from the droplets, and returns the particles to their original size. The output 170 is a particle enriched flow which is ready for sampling.

In an embodiment, the entire system including the pumps and the chiller weighs less than 30 kg and occupies a space less than 40 cm wide by 60 cm deep by 150 cm high.

The performance of this system and its components have been thoroughly tested. Test techniques have included collection of particles, fluorescence analysis of the collected particles, and others.

One test technique has been to detect monodisperse fluorescent particles by collection and fluorescence analysis. Another has included detection of other materials. It was found that particle losses are less than 10% independent of particle diameter for minor flows up 1 ½ liters per minute. The standard deviation for the virtual impactor was 1.8, 1.25 and 2.32 for minor flows of 1.5 L per minute (minor flow ratio 0.05) 1 L per minute (ratio 0.033) and 0.6 L per minute (ratio 0.2) respectively. It was also found that particle volatility does not influence the amount of concentration of the aerosols.

In the embodiment, the controller 118 may control the heating element, the war or control by the peristaltic pump 106, the amount of cooling by the recirculating chiller 140, and may also maintain information for example when the silica gel needs recharging.

The general structure and techniques, and more specific embodiments which can be used to effect different ways of carrying out the more general goals are described herein.

Although only a few embodiments have been disclosed in detail above, other embodiments are possible and the inventor(s) intend these to be encompassed within this specification. The specification describes specific examples to accomplish a more general goal that may be accomplished in another way. This disclosure is intended to be exemplary, and the claims are intended to cover any modification or alternative which might be predictable to a person having ordinary skill in the art. For example, the sizes given herein may differ, and additional or other parts may be used.

Also, the inventor(s) intend that only those claims which use the words “means for” are intended to be interpreted under 35 USC 112, sixth paragraph. Moreover, no limitations from the specification are intended to be read into any claims, unless those limitations are expressly included in the claims.

The controller described herein may be any kind of computer, either general purpose, or some specific purpose computer such as a workstation or formed of dedicated logic or a configurable logic block. The computer may be a Pentium class computer, running Windows XP or Linux, or may be a Macintosh computer. The programs may be written in C, or Java, or any other programming language. The programs may be resident on a storage medium, e.g., magnetic or optical, e.g. the computer hard drive, a removable disk or other removable medium. The programs may also be run over a network, for example, with a server or other machine sending signals to the local machine, which allows the local machine to carry out the operations described herein.

What is claimed is:

1. A particle detector, comprising:
a particle inlet;
a saturator and condenser, in communication with the particle inlet, and humidifying particles in the particle inlet, and cooling the humidifying particles using a semiconductor based cooler to increase a size of the particles;
a virtual impactor, separating the increased-in-size particles into a minor flow in which a percentage of particles is substantially increased, and a major flow, in which the percentage of particles is substantially decreased; and
a dryer, which dries the particles to return the particles to their original size.

2. A detector as in claim 1, wherein said the saturator and condenser each include a top portion and a bottom portion, and a U shaped conduit extending between the bottom portion of the saturator and the bottom portion of the condenser.

3. A detector as in claim 2, further comprising a gravity-fed drain at a bottommost portion of the U shaped portion.

4. A detector as in claim 3, wherein said drain forms a substantially closed system drain.

5. A detector as in claim 1, wherein said saturator comprises a sponge and a water supply which uses a pump to wet said sponge, and where the particle inlet extends to an area which passes through said sponge.

6. A detector as in claim 5, further comprising a heating element, in communication with said sponge, and maintaining said sponge at a specified temperature.

7. A detector as in claim 1, wherein said condenser comprises a recirculating chiller.

8. A detector as in claim 1, wherein said condenser maintains a specified surface at substantially =1° C.

9. A detector as in claim 1, wherein.said dryer includes drying beads.

10. A detector as in claim 1, wherein said condenser uses a thermoelectric cooler.

11. A method of detecting particles, comprising:
Receiving a stream of particles;
supersaturating in water vapor and cooling the particles, to increase a size of the particles, wherein said cooling includes using a semiconductor cooler to cool to a temperature close to freezing;
maintaining said temperature using a temperature probe to control a temperature of said semiconductor cooler;
concentrating the increased-in-size particles by inertial virtual impaction, and
returning the particles to their original size by diffusion and drying.

12. A method as in claim 11, further comprising gravity draining water in an area of said supersaturating.

13. A method as in claim 12, wherein said gravity draining comprises placing a drain in common for both areas of said supersaturation and cooling.

14. A method as in claim 13, wherein said draining comprises using a pressure sealed drain.

15. A method as in claim 11, wherein said saturating comprises a sponge and a water supply which uses a pump to wet said sponge, and where the particle inlet extends to an area which passes through said sponge.

16. A method as in claim 15, further comprising heating said sponge, and maintaining said sponge at a specified temperature.

17. A method as in claim 11, wherein said chilling comprises recirculating a cooling fluid.

18. A method as in claim 11, wherein said chilling comprises maintaining a specified surface at substantially −1° C.

19. A method as in claim 11, wherein said dryer includes drying beads.

20. A method as in claim 11, wherein said cooling uses a thermoelectric cooler.
